(19) United States
(12) Patent Application Publication
(10) Pub. No.: US 2026/0000444 A1
(43) Pub. Date: Jan. 1, 2026

(54) ULTRASONIC TREATMENT TOOL

(71) Applicant: OLYMPUS MEDICAL SYSTEMS CORP., Tokyo (JP)

(72) Inventors: Marina ASAHINA, Sagamihara-shi (JP); Tsubasa NIIYAMA, Tokyo (JP)

(21) Appl. No.: 18/990,546

(22) Filed: Dec. 20, 2024

Related U.S. Application Data
(63) Continuation of application No. PCT/JP2024/023323, filed on Jun. 27, 2024.

(51) Int. Cl. A61B 18/14; A61B 18/00

(52) U.S. Cl. CPC A61B 18/1445; A61B 2018/00077; A61B 2018/00095; A61B 2018/00101; A61B 2018/00994

(57) ABSTRACT

An ultrasonic treatment tool includes: a vibration transmission portion having a distal end provided with a treatment portion configured to treat a treatment target, the vibration transmission portion being configured to transmit ultrasonic vibration from a proximal end toward the treatment portion of the vibration transmission portion; a holding portion configured to be opened from and closed to the treatment portion; and a resin pad that is held to the holding portion and has a gripping face to grip the treatment target between the resin pad and the treatment portion.

### CROSS REFERENCES TO RELATED APPLICATIONS

This application is a continuation of International Application No. PCT/JP2024/023323, filed on Jun. 27, 2024, the entire contents of which are incorporated herein by reference.

### BACKGROUND

### 1. Technical Field

The present disclosure relates to an ultrasonic treatment tool.

### 2. Related Art

In the related art, there has been known an ultrasonic treatment tool that applies ultrasonic energy to a site to be treated (hereinafter, described as a target site) in a living tissue to treat the target site (see, for example, WO 2022/185406 A).

The ultrasonic treatment tool described in WO 2022/185406 A includes a rod member, a holder member, and a pad member (hereinafter, referred to as a resin pad) described below. The rod member includes a treatment portion that treats a living tissue at the distal end, to transmit ultrasonic vibration from the proximal end toward the treatment portion. The holder member is opened from and closed to the treatment portion. The resin pad is held to the holder member, and has a contact face that grips the living tissue between the contact face and the treatment portion.

There is a problem that, for example, when ultrasonic vibration is applied from a treatment portion after treatment (incision) of a target site is completed, the resin pad deteriorates due to frictional heat and cannot exhibit a desired function in some cases. On the other hand, the ultrasonic treatment tool described in WO 2022/185406 A includes a heat transfer member that transfers heat of the resin pad to the holder member, and reduces frictional heat of the resin pad.

### SUMMARY

In some embodiments, an ultrasonic treatment tool includes: a vibration transmission portion having a distal end provided with a treatment portion configured to treat a treatment target, the vibration transmission portion being configured to transmit ultrasonic vibration from a proximal end toward the treatment portion of the vibration transmission portion; a holding portion configured to be opened from and closed to the treatment portion; and a resin pad that is held to the holding portion and has a gripping face to grip the treatment target between the resin pad and the treatment portion, the resin pad including a first region including a gripping position at which the treatment target is gripped, the first region being a region in which heat generated by a treatment is large, and a second region other than the first region, the holding portion including a third region facing the first region and a fourth region facing the second region, the holding portion being configured such that a heat capacity per unit length of the third region is larger than a heat capacity per unit length of the fourth region, the holding portion including a jaw body configured to hold the resin pad, and the jaw body including a body portion to which the resin pad is attached, and an outer shell provided on an outer surface different from an outer surface to which the resin pad is attached, of outer surfaces of the body portion, the outer shell being made of a heat insulating material.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a view illustrating an ultrasonic treatment tool according to an embodiment;

FIG. 2 is a view illustrating a distal end portion of an ultrasonic treatment tool;

FIG. 3 is a view illustrating a distal end portion of the ultrasonic treatment tool;

FIG. 4 is a view illustrating a jaw;

FIG. 5 is a view illustrating a jaw;

FIG. 6 is a view illustrating an arm;

FIG. 7 is a view illustrating a wiper jaw;

FIG. 8 is a view illustrating a wiper jaw;

FIG. 9 is a diagram for describing an example of heat received by the resin pad in the longitudinal direction;

FIG. 10 is a partial cross-sectional view illustrating a distal end portion of a jaw;

FIG. 11 is a cross-sectional view of a jaw distal end portion taken along line A-A illustrated in FIG. 10;

FIG. 12 is a cross-sectional view of a jaw distal end portion taken along line B-B illustrated in FIG. 10; and

FIG. 13 is a view illustrating an example of a configuration of a conventional jaw distal end portion.

### DETAILED DESCRIPTION

Hereinafter, modes for carrying out the embodiments will be described with reference to the drawings. Note that the disclosure is not limited by the embodiments described below. Further, in the description of the drawings, the same parts will be described with the same reference numerals.

### Embodiments

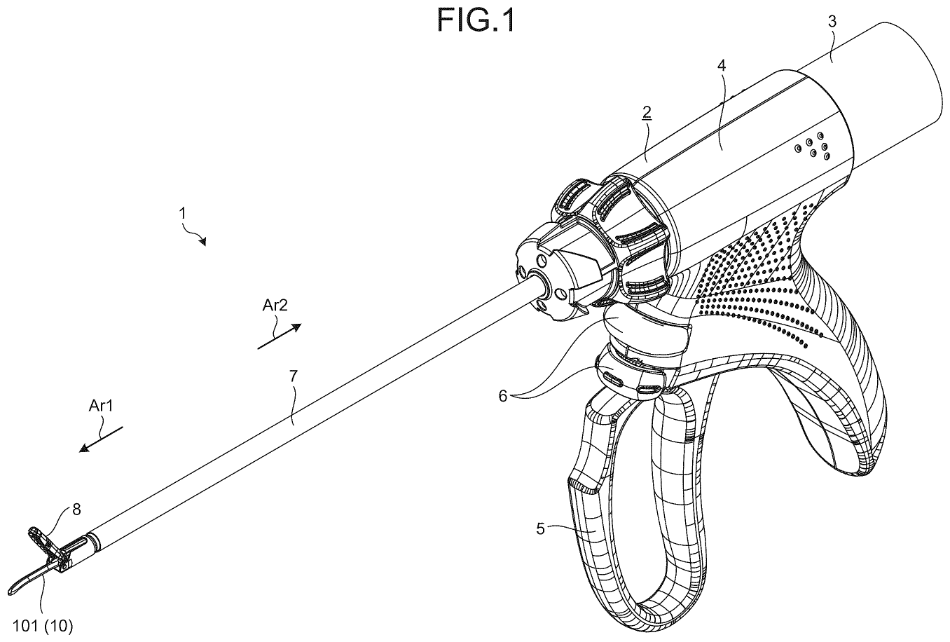

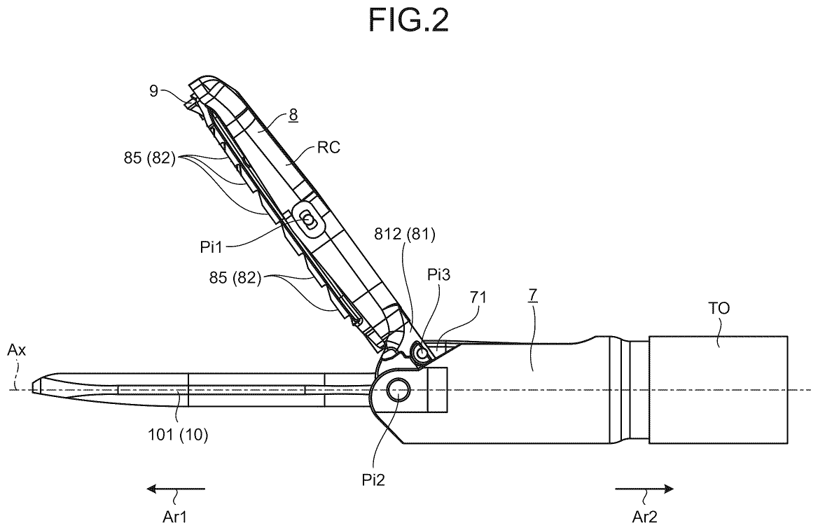

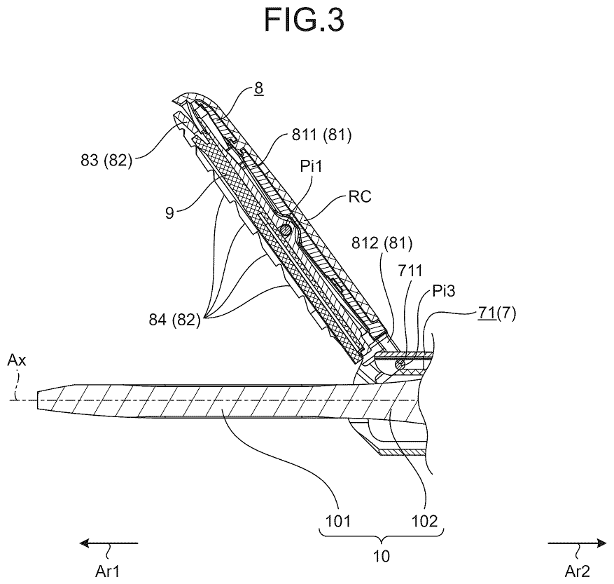

FIG. 1 is a diagram illustrating an ultrasonic treatment tool 1 according to an embodiment. FIGS. 2 and 3 are views illustrating a distal end portion of the ultrasonic treatment tool 1. Specifically, FIG. 2 is a view of the distal end portion of the ultrasonic treatment tool 1 viewed in a normal direction of a plane including the center axis Ax of a sheath 7 in a state where a jaw 8 and a vibration transmission portion 10 are included in the plane. FIG. 2 is a cross-sectional view of the distal end portion of the ultrasonic treatment tool 1 cut by a plane including the center axis Ax of the sheath 7 in a state where the jaw 8 and the vibration transmission portion 10 are included in the plane. The “width direction” described below means a direction orthogonal to the paper surface of each of FIGS. 2 and 3.

The ultrasonic treatment tool 1 applies ultrasonic energy and/or high frequency energy to a site to be treated (hereinafter, described as a target site) in a living tissue to treat the target site. Here, the treatment means, for example, coagulation and incision of a target site. As illustrated in FIG. 1, the ultrasonic treatment tool 1 includes a handpiece 2 and an ultrasonic transducer 3.

As illustrated in FIGS. 1 to 3, the handpiece 2 includes a holding case 4 (see FIG. 1), an operation handle 5 (see FIG. 1), a switch 6 (see FIG. 1), the sheath 7, the jaw 8, a resin pad 9 (see FIGS. 2 and 3), and a vibration transmission portion 10.

The holding case 4 supports the entire ultrasonic treatment tool 1.

The operation handle 5 is movably attached to the holding case 4 and receives an opening and closing operation by an operator.

The switch 6 is provided in a state of being exposed to the outside of the holding case 4, and receives an output start operation by the operator. Then, the switch 6 outputs an operation signal corresponding to the output start operation to a control device (not illustrated) electrically connected to the ultrasonic treatment tool 1.

The sheath 7 has a substantially cylindrical shape as a whole. Hereinafter, one side of the sheath 7 along the center axis Ax is referred to as a distal end side Ar1 (see FIGS. 1 to 3), and the other side is referred to as a proximal end side Ar2 (see FIGS. 1 to 3). The sheath 7 is attached to the holding case 4 by inserting part of the proximal end side Ar2 into the holding case 4 from the distal end side Ar1 of the holding case 4.

In the sheath 7, the outer peripheral face is covered with an electrically insulating outer tube TO.

In the sheath 7, the inner peripheral face is covered with an electrically insulating inner tube.

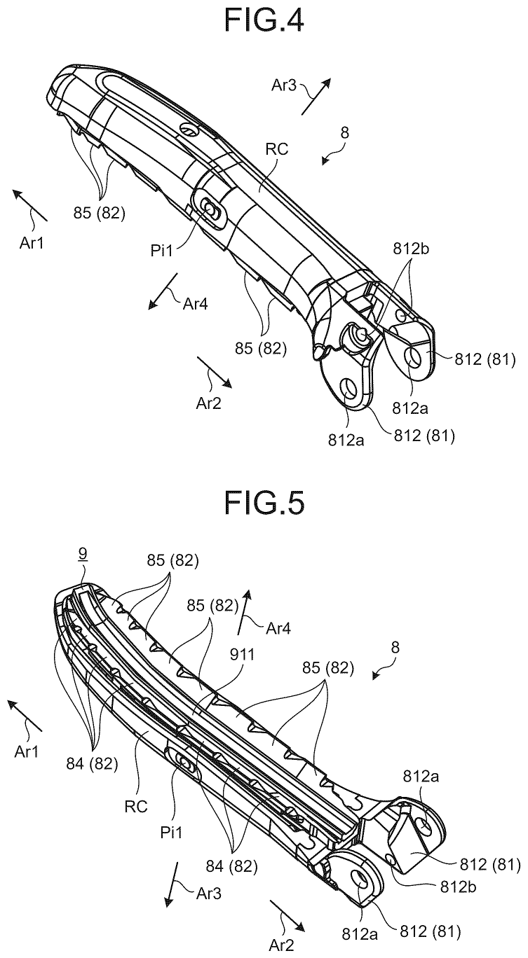

Note that, in the following description of the configuration of the jaw 8 and the resin pad 9, a side away from a treatment portion 101 of the vibration transmission portion 10 will be referred to as a back face side Ar3 (see FIGS. 4 to 9), and a side close to the treatment portion 101 will be referred to as a treatment portion side Ar4 (see FIGS. 4 to 9).

FIGS. 4 and 5 are views illustrating the jaw 8. Specifically, FIG. 4 is a perspective view of the jaw 8 when viewed from the back face side Ar3. FIG. 5 is a perspective view of the jaw 8 when viewed from the treatment portion side Ar4. FIG. 5 illustrates a state in which the resin pad 9 is assembled to the jaw 8.

The jaw 8 corresponds to a holding portion. The jaw 8 can be opened from and closed to the treatment portion 101 (see FIGS. 2 and 3) by being pivotally supported by the end portion of the sheath 7 at the distal end side Ar1. When the jaw 8 is closed to the treatment portion 101, the target site is gripped between the resin pad 9 held by the jaw 8 and the treatment portion 101. As shown in FIG. 4 or 5, the jaw 8 includes an arm 81 and a wiper jaw 82.

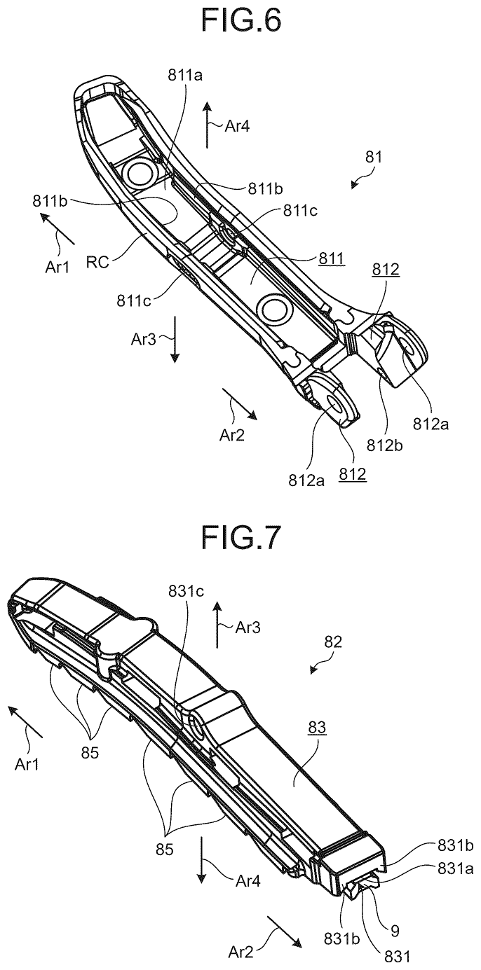

FIG. 6 is a view illustrating the arm 81. Specifically, FIG. 6 is a perspective view of the arm 81 when viewed from the treatment portion side Ar4.

The arm 81 is made of an electrically conductive material. As illustrated in FIG. 6, the arm 81 is a member in which an arm body 811 and a pair of bearing portions 812 are integrally formed.

The arm body 811 is configured by an elongated substantially plate body. In the present embodiment, the longitudinal direction of the arm body 811 is a direction along a curve toward the left side as it goes toward the distal end side Ar1 from the proximal end side Ar2 in a state where the jaw 8 is located above the treatment portion 101.

In the arm body 811, as illustrated in FIG. 6, a first recess 811a extending from the proximal end toward the distal end side Ar1 along the longitudinal direction of the arm body 811 is provided at the face on the treatment portion side Ar4.

As illustrated in FIG. 6, a first insertion hole 811c that penetrate in the width direction and through which a first pin Pi1 (see FIGS. 4 and 5) is inserted is provided in each of side wall portions 811b of the arm body 811 constituting the first recess 811a on both sides in the width direction. The two first insertion holes 811c are located substantially at the center of the arm body 811 in the longitudinal direction. A straight line connecting the two first insertion holes 811c is parallel to the width direction. In the present embodiment, the first pin Pi1 is fixed to the arm body 811 by welding with inserted into each first insertion hole 811c.

Further, an electrically insulating resin cover RC (see FIGS. 4 to 6) is integrally formed on the face of the arm body 811 at the back face side Ar3 in a state of covering the face of the back face side Ar3. In the present embodiment, the resin cover RC is insert-molded into the arm body 811, but the embodiment is not limited thereto. For example, a configuration in which the resin cover RC is fixed to the arm body 811 by snap-fitting or a metal pin may be used.

Each of the pair of bearing portions 812 is provided at the proximal end of the arm body 811, and is constituted by plate bodies facing each other in the width direction.

As illustrated in FIG. 6, each of the pair of bearing portions 812 is provided with a second insertion hole 812a that penetrates the front and back faces and through which each of the two second pins Pi2 is inserted. That is, the arm 81 is connected to the sheath 7 by the two second pins Pi2.

As illustrated in FIG. 4, each of the pair of bearing portions 812 is provided with a third insertion hole 812b which penetrates the front and back faces and through which a third pin Pi3 is inserted. In the present embodiment, the third pin Pi3 is fixed to the arm 81 by welding in a state of being inserted into each fourth insertion hole 711 (see FIG. 3) of an opening and closing mechanism 71 constituting the sheath 7 and each the third insertion hole 812b. That is, the arm 81 is connected to the opening and closing mechanism 71 by the third pin Pi3. Then, the arm 81 rotates about the two second pins Pi2 in conjunction with the movement of the opening and closing mechanism 71 to the distal end side Ar1 or the proximal end side Ar2 according to the opening and closing operation on the operation handle 5 by the operator. As a result, the jaw 8 is opened from and closed to the treatment portion 101.

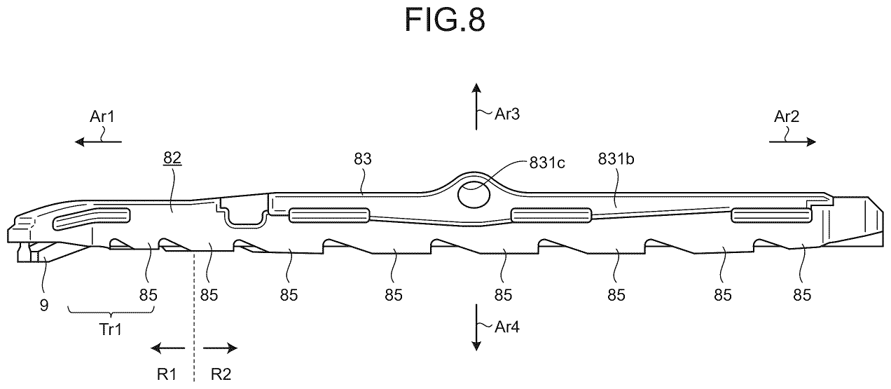

FIGS. 7 and 8 are views illustrating the wiper jaw 82. Specifically, FIG. 7 is a perspective view of the wiper jaw 82 when viewed from the back face side Ar3. FIG. 8 is a view of the wiper jaw 82 when viewed in the width direction. FIGS. 7 and 8 illustrate a state in which the resin pad 9 is assembled to the wiper jaw 82.

The wiper jaw 82 is made of an electrically conductive material such as stainless steel or a titanium alloy, and is attached to the arm 81. As shown in FIG. 7 or FIG. 8, the wiper jaw 82 includes a wiper jaw body 83, a plurality of first tooth portions 84 (see FIG. 3), and a plurality of second tooth portions 85.

The wiper jaw body 83 is configured by an elongated plate body extending along the longitudinal direction of the arm body 811. The outer shape of the wiper jaw body 83 is set to be substantially the same as the inner face shape of the first recess 811a. The wiper jaw body 83 is then disposed in the first recess 811a.

The wiper jaw body 83 includes a second recess 831 that penetrates from the proximal end to the distal end along the longitudinal direction of the wiper jaw body 83 and where the resin pad 9 is disposed is provided on the face on the treatment portion side Ar4. Hereinafter, the bottom face of the second recess 831 is referred to as a bottom face 831a, and side wall portions on both sides of the second recess 831 in the width direction are referred to as side wall portions 831b.

As illustrated in FIG. 7 or 8, the wiper jaw body 83 is provided with a fifth insertion hole 831c which penetrates from one side wall portion 831b to the other side wall portion 831b along the width direction and through which the first pin Pi1 is inserted. The fifth insertion hole 831c is located substantially at the center of the wiper jaw body 83 in the longitudinal direction. The center axis of the fifth insertion hole 831c is parallel to the width direction. The wiper jaw body 83 is pivotally supported by the arm 81 so as to be swingable about the first pin Pi1. That is, by making the wiper jaw 82 swingable about the first pin Pi1, the position where the strongest force is applied to the target site when the target site is gripped between the jaw 8 and the treatment portion 101 is positioned at substantially the center of the jaw 8 in the longitudinal direction instead of the proximal end side Ar2 of the jaw 8. As a result, a force is substantially uniformly applied to the target site gripped between the jaw 8 and the treatment portion 101.

The plurality of first tooth portions 84 protrudes from the one side wall portion 831b toward the treatment portion side Ar4, and is disposed side by side along the longitudinal direction of the wiper jaw body 83.

The plurality of second tooth portions 85 protrudes from the other side wall portion 831b toward the treatment portion side Ar4, and is disposed side by side along the longitudinal direction of the wiper jaw body 83.

The plurality of first tooth portions 84 and the plurality of second tooth portions 85 are provided in a state where the resin pad 9 is sandwiched in a state where the resin pad 9 is attached to the wiper jaw 82.

The resin pad 9 is softer than the vibration transmission portion 10, is made of a resin material having electrical insulation and biocompatibility, for example, polytetrafluoroethylene (PTFE), and has a substantially rectangular parallelepiped shape extending along the longitudinal direction of the arm body 811. In addition, the resin pad 9 has a third recess 91 (see FIG. 10) extending from the proximal end toward the distal end side Ar1 is provided on the face on the treatment portion side Ar4. Further, the resin pad 9 is disposed inside the second recess 831. When the jaw 8 is brought close to the treatment portion 101, a bottom face 911 of the third recess 91 of the resin pad 9 contacts the treatment portion 101. The bottom face 911 has a substantially flat shape. The bottom face 911 corresponds to a gripping face. Hereinafter, for convenience of description, the bottom face 911 is referred to as a gripping face 911.

The vibration transmission portion 10 has an elongated shape and is made of an electrically conductive material. Then, as illustrated in FIG. 2 or 3, the vibration transmission portion 10 is inserted into the sheath 7 in a state where the treatment portion 101 is exposed to the outside. The vibration transmission portion 10 includes the treatment portion 101 and a shaft 102.

The treatment portion 101 is provided at the distal end of the shaft 102. As in the jaw 8, the treatment portion 101 extends along a curve toward the left side as it goes toward the distal end side Ar1 when viewed from the proximal end side Ar2 in a state where the jaw 8 is located above.

The shaft 102 has an elongated shape extending along the center axis Ax, and an end portion on the proximal end side Ar2 is connected to a bolted Langevin type transducer (BLT) constituting the ultrasonic transducer 3. Then, the shaft 102 transmits the ultrasonic vibration generated by the BLT from the end portion of the proximal end side Ar2 to the treatment portion 101. In the present embodiment, the ultrasonic vibration is longitudinal vibration that vibrates in a direction along the center axis Ax. At this time, the treatment portion 101 vibrates with a desired amplitude by the longitudinal vibration of the vibration transmission portion 10.

An annular lining having electrical insulation and elasticity and extending along the circumferential direction around the center axis of the shaft 102 is attached to the outer peripheral face of the shaft 102 described above. The lining is disposed at each node of the longitudinal vibration of the vibration transmission portion 10. The lining contacts the inner tube in a state where the vibration transmission portion 10 is inserted into the sheath 7. The inner tube has a function of securing electrical insulation between the sheath 7 and the vibration transmission portion 10. The lining has a function of sealing liquid that has entered the gap between the inner tube and the vibration transmission portion 10.

The ultrasonic transducer 3 is detachably connected to an end portion of the holding case 4 on the proximal end side Ar2. Although not specifically illustrated, the ultrasonic transducer 3 includes a BLT that generates ultrasonic vibration in response to supply of AC power.

The control device (not illustrated) electrically connected to the ultrasonic treatment tool 1 described above controls the operation of the ultrasonic treatment tool 1 as described below according to the operation signal from the switch 6.

The control device supplies high frequency power between the jaw 8 and the treatment portion 101 via the sheath 7 and the shaft 102. Then, a high-frequency current flows between the treatment portion 101 and the plurality of tooth portions (the first tooth portion 84 and the second tooth portion 85) having the same potential. That is, the high-frequency current flows through the target site gripped between the jaw 8 and the treatment portion 101. In other words, high frequency energy is applied to the target site.

Furthermore, the control device supplies AC power to the BLT constituting the ultrasonic transducer 3 to generate ultrasonic vibration in the BLT. Then, ultrasonic vibration is applied from the treatment portion 101 to the target site gripped between the jaw 8 and the treatment portion 101. In other words, ultrasonic energy is applied to the target site.

Then, Joule heat is generated in the target site by the flow of the high-frequency current. In addition, frictional heat is generated between the treatment portion 101 and the target site by the vertical vibration of the treatment portion 101. As a result, the target site is incised while coagulating.

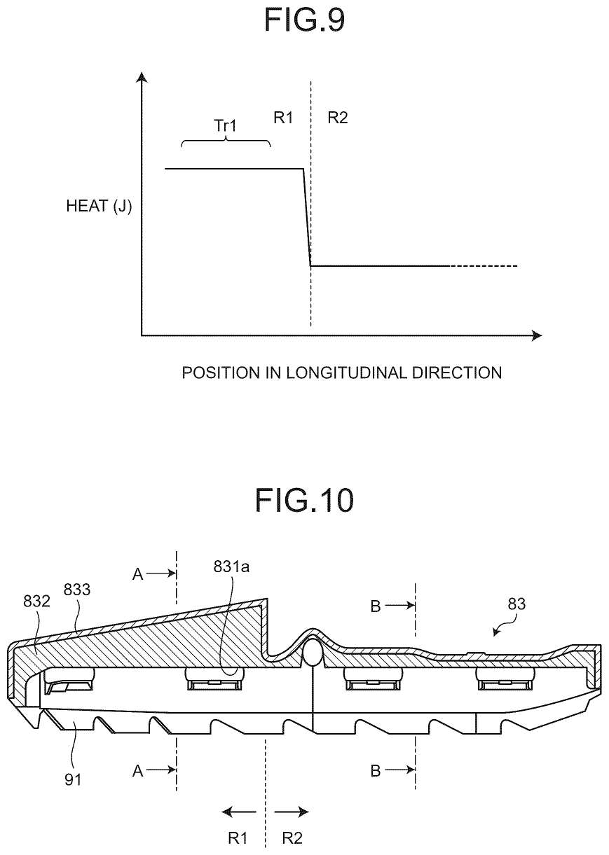

Here, FIG. 9 is a diagram for describing an example of heat received by the resin pad 9 in the longitudinal direction (center axis Ax direction). Here, an example in which a target site is gripped in a gripping region Tr1 (see FIG. 8) of the wiper jaw 82 on the distal end side Ar1 relative to the first pin Pi1 and ultrasonic energy is applied to the target site will be described. As illustrated in FIG. 9, heat (J) due to frictional heat received by the resin pad 9 increases in the gripping region Tr1 and around thereof. At this time, the distal end side Ar1 is set to a high temperature region R1 (first region), and the proximal end side Ar2 is set to a low temperature region R2 (second region) with a rising position where the heat received by the resin pad 9 rises in the longitudinal direction (center axis Ax direction) of the wiper jaw 82 as a boundary.

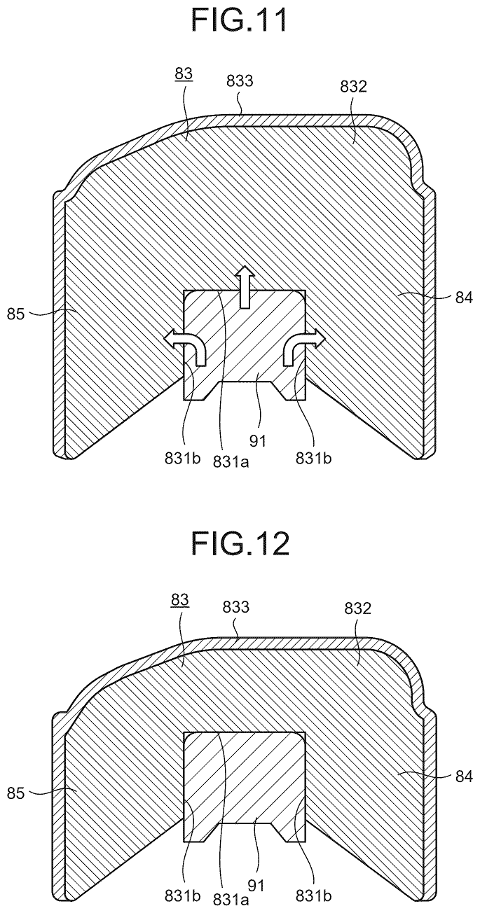

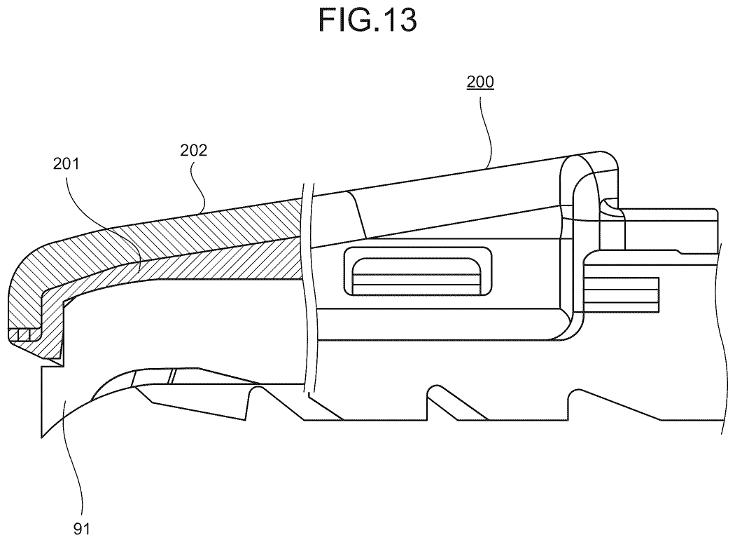

FIG. 10 is a partial cross-sectional view showing the distal end portion of the jaw. FIG. 11 is a cross-sectional view of the jaw distal end portion taken along line A-A illustrated in FIG. 10. FIG. 12 is a cross-sectional view of the jaw distal end portion taken along line B-B illustrated in FIG. 10. As illustrated in FIG. 10, the wiper jaw body 83 includes a body portion 832 and an outer shell 833 formed on an outer surface of the body portion 832. In the present embodiment, the outer shell 833 is formed on the outer surface on a side on which the resin pad 9 is not provided and on which the arm 81 is provided. In the present embodiment, the body portion 832 is made of the same material as a whole.

In the wiper jaw body 83, a region (third region: see FIG. 11) facing the high temperature region R1 of the resin pad 9 has a larger cross-sectional area (volume) of the body portion 832 than a region (fourth region: see FIG. 12) facing the low temperature region R2 of the resin pad 9. Here, for example, the “facing region” refers to a region of the wiper jaw body 83 corresponding to the region of the resin pad 9 in the longitudinal direction (the center axis Ax direction).

As a result, in the wiper jaw body 83, the heat capacity per unit length of the region corresponding to the high temperature region R1 is larger than the heat capacity per unit length of the region corresponding to the low temperature region R2. Here, the “unit length” refers to a predetermined length of the wiper jaw body 83 in the longitudinal direction (center axis Ax direction).

By increasing the heat capacity per unit length of the region corresponding to the high temperature region R1, the wiper jaw body 83 absorbs a large amount of heat received by the resin pad 9 (see the arrow in FIG. 11).

FIG. 13 is a view illustrating an example of a configuration of a conventional jaw distal end portion. A conventional wiper jaw body 200 includes a body portion 201 and a cover 202 formed on an outer surface of the body portion 201. At this time, the cover 202 is thicker than the outer shell 833, and the difference in volume per unit length in the body portion 201 is small in the longitudinal direction (corresponding to the center axis Ax direction) of the wiper jaw body 200. For this reason, in the conventional wiper jaw body 200, the difference in heat capacity between the high temperature region and the low temperature region, the heat capacity corresponding to the heat received by the resin pad 9, is small.

According to the present embodiment described above, the following effects are obtained.

In the wiper jaw body 83 of the ultrasonic treatment tool 1 according to the present embodiment, the heat capacity at the position corresponding to the high temperature region R1 where heat is generated by treatment is made larger than the heat capacity at the position corresponding to the low temperature region R2 where the heat received by the resin pad 9 is small, the high temperature region R1 including the gripping region Tr1 for the target site of the resin pad 9.

Therefore, as compared with the conventional body portion 201 having a small difference in heat capacity in the longitudinal direction, frictional heat generated in the resin pad 9 (gripping face 911) by application of ultrasonic vibration can be efficiently received by (transferred to) the wiper jaw body 83.

Therefore, according to the ultrasonic treatment tool 1 according to the present embodiment, it is possible to suppress deterioration of the resin pad 9 while suppressing an increase in size.

In the embodiment, the example in which the gripping region Tr1 is located at the distal end of the resin pad 9 and the portion at the proximal end relative to the distal end is the low temperature region R2 is described, but the embodiment is not limited thereto. For example, in a case where the resin pad 9 grips the target site at the proximal end relative to the distal end, the distal end is the low temperature region, and the heat capacity (volume) is adjusted accordingly. In this case, in the wiper jaw body 83, the volume of the distal end portion is smaller than the volume at the proximal end portion. A method of dividing the region is not limited to dividing into two regions, for example, the gripping region Tr1 is located at the center of the resin pad 9 in the longitudinal direction, and the region is divided into a low temperature region, a high temperature region, and a low temperature region in order from the distal end in the longitudinal direction of the resin pad 9.

In the embodiment, an example is described in which the heat capacity is increased by increasing the volume of the region of the body portion 832 corresponding to the high temperature region R1 to increase the amount of heat received by the wiper jaw body 83, but the specific heat may be partially changed without changing the volume of the wiper jaw body 83 in the longitudinal direction (the center axis Ax direction). Specifically, the body portion 832 in the region (third region) corresponding to the high temperature region R2 may be made of a material having a high thermal conductivity, and specific heat of the third region may be larger than specific heat of the fourth region. Examples of the material having high thermal conductivity include polyether ether ketone (PEEK) and polyimide (PI). Such partial modification of the material may in fact be used for the purpose of increasing the thermal efficiency at the distal end or the proximal end of the wiper jaw body 83, in terms of configuration.

In the present embodiment, the outer shell 833 may be made of a heat insulating material that shields heat. By imparting a heat insulating effect to the outer shell 833, heat transfer of heat received by the wiper jaw body 83 from the resin pad 9 to the arm 81 is suppressed. As a result, it is possible to suppress the thermal influence of the heat of the arm 81 on portions other than the target site.

The wiper jaw body 83 may have only the body portion 832 without the outer shell 833.

### Other Embodiments

In the above-described embodiment, the configuration in which both the ultrasonic energy and the high frequency energy are applied to the target site is used, but the embodiment is not limited thereto. For example, a configuration of applying only ultrasonic energy to a target site or a configuration of applying at least one of high frequency energy and thermal energy in addition to ultrasonic energy to a target site may be used. Here, applying thermal energy to the target site means transferring heat of a heater or the like to the target site. In the configuration in which only ultrasonic energy is applied, the wiper jaw 82 does not need to be made of an electrically conductive material.

Further, in the above-described embodiment, as the holding portion, a configuration including the arm 81 and the wiper jaw 82 swingably attached to the arm 81 is used, but the embodiment is not limited thereto. The holding portion is only required to be opened from and closed to the treatment portion 101, and a configuration in which the wiper jaw 82 is fixed to the arm 81 and does not swing may be used.

As described above, the ultrasonic treatment tool according to the disclosure is useful for suppressing deterioration of the resin pad while suppressing an increase in size.

According to the ultrasonic treatment tool of the disclosure, it is possible to suppress deterioration of the resin pad while suppressing an increase in size.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic treatment tool comprising:
a vibration transmission portion having a distal end provided with a treatment portion configured to treat a treatment target, the vibration transmission portion being configured to transmit ultrasonic vibration from a proximal end toward the treatment portion of the vibration transmission portion;
a holding portion configured to be opened from and closed to the treatment portion; and
a resin pad that is held to the holding portion and has a gripping face to grip the treatment target between the resin pad and the treatment portion,
the resin pad including
a first region including a gripping position at which the treatment target is gripped, the first region being a region in which heat generated by a treatment is large, and
a second region other than the first region,
the holding portion including a third region facing the first region and a fourth region facing the second region, the holding portion being configured such that a heat capacity per unit length of the third region is larger than a heat capacity per unit length of the fourth region,
the holding portion including a jaw body configured to hold the resin pad, and
the jaw body including
a body portion to which the resin pad is attached, and
an outer shell provided on an outer surface different from an outer surface to which the resin pad is attached, of outer surfaces of the body portion, the outer shell being made of a heat insulating material.

2. The ultrasonic treatment tool according to claim 1, wherein
in the holding portion, a volume per unit length of the third region is larger than a volume per unit length of the fourth region.

3. The ultrasonic treatment tool according to claim 1, wherein
in the holding portion, a specific heat of the third region is larger than a specific heat of the fourth region.

4. The ultrasonic treatment tool according to claim 1, wherein
the first region is distally disposed, compared with the second region.

5. The ultrasonic treatment tool according to claim 4, wherein
a volume per unit length of the body portion is larger than a volume per unit length of the outer shell.

6. The ultrasonic treatment tool according to claim 1, wherein
the holding portion is made of an electrically conductive material, and
the ultrasonic treatment tool is configured to supply high frequency power between the holding portion and the treatment portion to supply high frequency energy to the treatment target.
